(19) United States
(12) Patent Application Publication
(10) Pub. No.: US 2009/0104135 A1
(43) Pub. Date: Apr. 23, 2009

(54) Use of Mineral/Organic Composite Material in the Form of an Ultraviolet Radiation Protective Agent

(75) Inventors: Stephane Daniele, Villeurbanne (FR); Veronique Guyot-Ferreol, Paris (FR); Jean-Francois Tranchant, Marigny les Usages (FR); Eugene Hubert, St Andre De La Roche (FR); Caroline Hubert, St. Andre De La Roche (FR)

Correspondence Address: OLIFF & BERRIDGE, PLC, P.O. BOX 320850, ALEXANDRIA, VA, 22320-4850, US

(73) Assignees: LVMH RECHERCHE, ST. JEAN DE BRAYE (FR); CENTRE NATIONAL DE LA RECHERCHE SCIENTIFIQUE, PARIS (FR); UCBL UNIVERSITE CLAUDE BERNARD DE LYON 1, VILLEURBANNE (FR)

(21) Appl. No.: 11/990,006

(22) PCT Filed: Aug. 4, 2006
(86) PCT No.: PCT/FR2006/001904

§ 371 Date: Dec. 19, 2008

(30) Foreign Application Priority Data
Aug. 5, 2005 (FR) 05 08382

(51) Int. Cl. A61K 8/33; A61Q 17/04

(52) U.S. Cl. 424/60; 424/59

(57) ABSTRACT

The invention relates to the use of composite material which comprises nanoparticles of a least one metal derivative and at least one organic sunscreen agent which is chemically bound with said particles in a covalent manner in the form of an ultraviolet radiation protective agent containing the inventive composite material. Cosmetic ultraviolet radiation protective compositions comprises said mixture are also disclosed.

The invention relates to the use of a composite material, comprising nanoparticles of a metal derivative and organic sun filters, as a protective material against ultraviolet radiation. The invention also relates to cosmetic compositions which incorporate said composite material and are intended to protect the skin from ultraviolet radiation (UV).

Commercial sun creams aim to protect the skin against UV-B radiation (radiations between 290 nm and 320 nm) and UV-A radiation (320 nm to 400 nm) by means of the sun filters which they contain, since said radiations are responsible for erythemas and burning of the skin, as well as for accelerating the ageing of the skin, and indeed for certain cancers.

In fact, depending on the wavelength thereof, said radiations may penetrate deeply, reach the dermis and be able to provoke photo-toxic or photo-allergenic reactions, particularly in the case of people with light skin. Further, UV-A deteriorates the structural proteins of the skin, particularly the collagen and elastin fibres, thus reducing the tone and elasticity of the skin, and leads to the appearance of wrinkles in skin which is continually exposed to the rays of the sun. Finally, UV-A also causes melanomata through its mutagenic action.

Due to recent increase in awareness, the use of sun creams or sun screens with a high protection factor (SPF) which absorb the whole of the sun's UV-spectrum (total screening) is recommended. The most effective creams are composed of opaque inorganic oxides, and more specifically of titanium dioxide (TiO2) and zinc oxide (ZnO), combined with organic filters, such as for example para-aminobenzoic acid and derivatives of benzophenone and camphor, to modify and increase the protection factor (SPF) of the product.

However, there exist no organic filters which are effective against the greater UV wavelengths from 370 nm to 400 nm. Further, it has been found that some of these filters display oestrogenic activities in vitro, i.e. they comprise female hormones (M. Schlumpf et al., SOFW Journal, 127(7), (2001), pages 10-15; and Comment in: Environ. Health Perspec., 109(11), (2001), page A517).

Nowadays, the use of sun creams with ever higher protection factors leads to a gradual absorption of organic filters by the skin. Thus, some derivatives of benzophenone have been detected in human urine 4 hours after the application onto the skin of a sun cream which contained them. Some filters also become pollutants and are found in swimming water, in fish, and in human milk.

It should also be noted that current products with high photoprotective power consist of organic sun filters and of mixtures of particles of metal oxide, such as TiO₂. Titanium dioxide is a semiconductor material which, when it is irradiated with UV rays, can induce photocatalytic phenomena.

To overcome this problem, the particles of TiO₂ must therefore be photostabilised by a surface treatment, as described for example in the patent EP-B-0 461 130, where TiO₂ nanoparticles were treated with phosphate anions.

More generally, TiO₂ nanoparticles of which the surface has been covered with aluminium, or preferably with silica (see patent applications EP-A-0 518 772 and EP-A-O 518 773) are found in cosmetic products.

However, these systems do not solve the problem of the organic filters which can penetrate through the skin (M. Schlumpf et al. ibid.).

To overcome this problem, an encapsulation of organic sun filters in silica particles has been proposed, as described for example in patent applications WO 2003/011239 and WO 2002/078665 and the publications of N. Lapidot et al., Journal of Sol-Gel Science and Technology, 26 (1/2/3), (2003), pages 67-72, of F. Pflueker et al., SOFW Journal, 128(6), (2002), pages 24-26 and of C. Anselmi et al., International Journal of Pharmaceutics, 242(1-2), (2002), pages 207-211.

Still other solutions have been proposed to solve the problem of insufficient UV-A absorption, as, for example, by combining filters with a compound of zirconium (cf. FR 2 799 120).

However, all these approaches have numerous drawbacks, which include, on the one hand, treatments which are expensive and difficult to carry out, and on the other hand, an insufficient absorption of UV-A, since the organic filters and the silica do not absorb rays with a wavelength greater than 360 nm.

Therefore, there still remains a need for protective materials which are effective on a wider range of the UV spectrum, and effective in particular in protecting against UV-A rays, while limiting the penetration of organic filters through the skin.

Thus, a first object of the present invention is to provide materials for protection against UV rays which do not lead to the above-mentioned drawbacks.

In particular, it is an object of the present invention to provide a material which can absorb the majority of radiation from the UV-A and UV-B spectra, where said material is absorbed very little, or not at all, by the skin.

Another object is to provide a material which can absorb the majority of radiation from the UV-A and UV-B spectra and induces no photocatalytic phenomena, or few photocatalytic phenomena.

Another object is to prevent, or greatly reduce, the penetration of organic filters through the skin.

Yet another object is to provide a material which is not harmful, or of limited harm, to the environment.

The inventors have now discovered that, surprisingly, the objects described above are achieved, in whole or in part, by the use of the composite materials described in detail below.

Thus, in accordance with a first aspect, the invention relates to a composite material comprising:

nanoparticles of at least one metal derivative and

at least one organic sun-filter derivative, covalently chemically bound to said nanoparticles,

in the form of an ultraviolet ray protective agent.

Advantageously, said sun-filter derivative is covalently chemically bound to said metal derivative.

In said composite material, the metal derivative can absorb ultraviolet (UV) rays. Said metal derivative must further be able to take a form which allows one or more covalent bonds to be created with the organic filter which absorbs the UV.

The preferred metal derivatives are those which have semiconductor properties and are able to absorb ultraviolet rays. Metal derivatives which are toxic neither for humans nor for the environment are also preferred.

Thus, the metal of said metal derivative may advantageously be selected from the group consisting of titanium, zinc, cerium, zirconium, copper, and mixtures thereof, and preferably from the group consisting of titanium, cerium, zinc, and mixtures thereof. Most preferably, the metal is titanium.

According to an especially advantageous embodiment of the present invention, the metal derivative is a metal oxide or a mixture of metal oxides.

According to a preferred embodiment, said metal oxide and the metal oxides of said mixture are selected from the group consisting of titanium dioxide (TiO₂), zinc monoxide (ZnO), the cerium oxides (Ce₂O₃ and CeO₂), zirconium dioxide (ZrO₂) and the copper oxides (CuO and Cu₂O), and advantageously, titanium dioxide, zinc monoxide (ZnO) and the cerium oxides (Ce₂O₃ and CeO₂).

By nanoparticles are meant nanoparticles which have a mean diameter of between 2 nm and 50 nm, advantageously between 4 nm and 30 nm, preferably between 6 nm and 20 nm, and even more preferably between 8 nm and 10 nm.

Nanoparticles with a mean diameter of less than 2 nm are a possibility. However, when the mean diameter is less than the size of the pores of the dermis, the nanoparticles can go through the skin, which is not in accordance with a preferred embodiment of the present invention.

Also, nanoparticles of which the mean diameter is greater than 50 nm are nevertheless a possibility for the composite materials which can be used in the invention, but may lead to an undesirable visual effect, and specifically to a whitening effect on the skin, for example in the case where the selected metal is titanium, in oxidised form TiO₂.

The organic sun filter derivative originates from an “organic sun filter”, which blocks UV-B and UV-A rays and is selected from the organic sun filters known to the person skilled in the art. By “organic sun filter” is meant any organic compound which absorbs UV rays in the wavelength range generally of 250 nm to 400 nm, which range is not limiting, however.

According to a preferred embodiment of the present invention, the organic sun filter has the formula:

R″-A-Y

in which
R″ represents a sun-filter residue, comprising a group which absorbs ultraviolet rays in the wavelength range from 250 nm to 400 nm,
A-Y represents a carboxylic group COOY or sulphonic group SO₃Y, where Y is selected from:

a hydrogen atom;

a cation of an alkali metal of an alkaline-earth metal from the periodic table;

a cation derived from a primary, secondary, tertiary or quaternary amine, or else an ammonium cation; and

a saturated or unsaturated, linear, branched or cyclic hydrocarbon group, containing 1 to 30 carbon atoms.

The sun filters as defined above may contain one or more carboxylic acid and/or sulphonic acid functional groups, in the form of a salt or ester. Furthermore, organic sun filters are preferred in which the residue R″ comprises at least one aromatic ring, for example benzene and/or benzimidazole and/or at least one bornyl ring. Even more preferably, at least one carboxylic acid functional group and/or sulphonic acid functional group is carried on at least one aromatic ring.

Thus, the organic sun filters which can be used in the present invention are advantageously selected from the organic sun filters comprising at least one carboxylic acid functional group and/or at least one sulphonic acid functional group, optionally in the form of a salt or ester.

Examples include carboxylic acids and sulphonic acids, but the acids mentioned above also comprise other functional groups, such as natural or non-natural amino acids, aminosulphonic acids, carboxylic and/or sulphonic keto acids, carboxylic and/or sulphonic hydroxy acids, and others, which may be linear, branched, cyclic, saturated, unsaturated, and/or aromatic, these compounds further being known for their properties as agents which block/absorb ultraviolet rays, and/or having intrinsic, as yet unknown properties of agents which block/absorb ultraviolet rays.

Examples of suitable organic sun filters for the present invention include, but are not limited to, the following acids and the cosmetically acceptable salts or esters thereof:
4-aminobenzoic acid;
4-dialkylaminobenzbic acids of which the alkyl groups, optionally carrying a hydroxyl group, are C₁-C₈ alkyl groups;
2-phenylbenzimidazole-5-sulphonic acid;
3,3′-(1,4-phenylidenedimethylidene)-bis-(7,7-dimethyl-2-oxobicyclo[2,2,1]-hept-1-ylmethanesulphonic) acid or benzylidene-camphor sulphonic acid;
α-(oxo-2-bornylidene-3)-toluene-4-sulphonic acid or terephthalylidene-camphor sulphonic acid;
2-hydroxy-4-methoxybenzophenone-5-sulphonic acid;
2,2′-(1,4-phenylene)-bis-1H-benzimidazole-4,6-disulphonic acid;
salicylic acid
4-alkoxycinnamic acids of which the alkoxy group is a C₁-C₁₀ alkoxy group;
3,3′-carbonyl-bis-(4-hydroxy-6-methoxy)benzene sulphonic acid;
2-cyano-3,3-diphenylacrylic acid; and
anthranylic acid.

The preferred organic sun filters which are suitable for the present invention, in the form of an acid, salt or ester, are brought together in Table A below, where “CTFA” designates the “Cosmetic Toiletry and Fragrance Association”:

TABLE A
Preferred organic sun filters

| | | |
|---|---|---|
| CTFA name | Trade name | Nomenclature |

ACIDS and SALTS:

| | | |
|---|---|---|
| PABA | | 4-aminobenzoic acid |
| Phenylbenzimidazole | EUSOLEX 232 ® | 2-phenyl-benzidamole-5-sulphonic acid |
| Sulphonic Acid | | |
| Benzylidene | MEXORYL SL ® | (3,3′-(1,4-phenylidene- |
| Camphor Sulphonic | | dimethylidene)bis-(7,7-dimethyl-2- |
| Acid | | oxobicyclo[2,2,1]hept-1-yl-methane- |
| | | sulphonic) acid and the salts thereof |
| Terephthalylidene | MEXORYL SX ® | α-(oxo-2-bornylidene-3)-toluene-4- |
| Dicamphor Sulphonic | | sulphonic acid and the salts thereof |
| Acid | | |
| Benzophenone-4 | UVINUL MS 40 ® | 2-hydroxy-4-methoxy-benzophenone-5- |
| | | sulphonic acid |
| Benzophenone-5 | | Sodium salt of 2-hydroxy-4- |
| | | methoxybenzophenone-5-sulphonic |
| | | acid |
| Disodium Phenyl | NEO HELIOPAN | Disodium salt of 2,2′-(1,4-phenylene)- |
| Dibenzimidazole | AP ® | bis-1H-benzimidazole-4,6-disulphonic |
| Tetrasulphonate | | acid |
| Tea Salicylate | NEO HELIOPAN | Triethanolamine salicylate |
| | TYPE TS ® | |
| Dea-Methoxy- | | Diethanolamine salt of 4- |
| cinnamate | | methoxycinnamic acid |
| Benzophenone-9 | UVINUL DS 49 ® | Disodium salt of 3,3′-carbonyl-bis-(4- |
| | | hydroxy-6-methoxy)-benzene sulphonic |
| | | acid |

ESTERS:

| | | |
|---|---|---|
| Ethylhexyl | PARSOL MCX ® | 2-ethylhexyl 4-methoxycinnamate |
| Methoxycinnamate | | |
| Octocrylene | PARSOL 340 ® | 2-ethylhexyl 2-cyano-3,3- |
| | | diphenylacrylate |
| PEG-25 PABA | UVINUL P 25 ® | Ethoxylated ethyl 4-aminobenzoate |
| Ethylhexyl Dimethyl | ESCALOL 507 ® | 2-ethylhexyl 4-dimethylaminobenzoate |
| PABA | | |
| Homosalate | EUSOLEX HMS ® | 3,3,3-trimethylcyclohexyl salicylate |
| Isoamyl-p-Methoxy- | UVSOB 360 ® | Isopentyl 4-methoxycinnamate |
| cinnamate | | |
| Ethylhexyl Salicylate | ESCALOL 587 ® | 2-ethylhexyl salicylate |
| Ethyl Dihydroxy- | | Ethyl 4-[bis(2-hydroxypropyl)amino]- |
| propyl PABA | | benzoate |
| Menthyl Anthranylate | DERMOBLOCKMA ® | p-menth-3-yl anthranylate |
| Glyceryl PABA | ESCALOL 106 ® | Glyceryl 4-dimethylaminobenzoate |
| Cinoxate | GIV-TAN ® | 2-ethoxyethyl 4-methoxycinnamate |

Examples of organic sun filters which are even more preferred for the present invention include, in particular, the compounds sold under the trade names PABA (para-aminobenzoic acid), Uvinul MS-40® (5-benzoyl-4-hydroxy-2-methoxybenzenesulphonic acid), Eusolex 232® (2-phenylbenzimidazole-5-sulphonic acid), Mexoryl SL® (3,3′-(1,4-phenyldene-dimethylidene)bis-(7,7-dimethyl-2-oxobicyclo[2,2,1]hept-1-yl-methane-sulphonic) acid and the salts thereof), Mexoryl SX® (α-(oxo-2-bornylidene-3)-toluene-4-sulphonic acid and the salts thereof), Parsol 340® (2-ethylhexyl 2-cyano-3,3-diphenylacrylate), octyl 4-methoxycinnamate, Uvinul P-25 (ethoxylated ethyl 4-aminobenzoate).

The following sun filters are particularly preferred:

PABA or para-aminobenzoic acid;

Eusolex 232® or 2-phenylbenzimidazole-5-sulphonic acid; and

Uvinul MS-40® 5-benzoyl-4-hydroxy-2-methoxybenzenesulphonic acid.

The composite material which can be used within the scope of the present invention comprises at least one metal derivative, as has just been defined, chemically bound by covalent bonding with at least one derivative of an organic sun filter which blocks/absorbs UV rays.

By “sun filter derivative” is meant, in the present invention, a sun filter of which at least one of the reactive atoms necessary for the formation of the covalent bond is absent. Thus, for example, if the sun filter is a carboxylic or sulphonic acid with at least one carboxylic acid functional group COOY and/or at least one sulphonic acid functional group SO₃Y, the “derivative” corresponds to said sun filter with at least one atom Y being absent.

According to a preferred embodiment of the present invention, the composite material which can be used within the scope of the present invention comprises titanium dioxide nanoparticles which are chemically grafted, by covalent bonding, to at least one sun-filter derivative, by means of at least one carboxylic and/or sulphonic functional group.

Highly satisfactory results are obtained with titanium dioxide nanoparticles onto the surface of which solar filters, in particular those defined above, are grafted.

According to an especially preferred embodiment, the invention relates to the use of a composite material comprising titanium dioxide nanoparticles which are chemically grafted, by covalent bonding, to at least one sun-filter derivative, by means of at least one carboxylic and/or sulphonic functional group, said sun filter being selected from the groups consisting of para-aminobenzoic acid, 5-benzoyl-4-hydroxy-2-methoxybenzenesulphonic acid, 2-phenylbenzimidazole-5-sulphonic acid, as well as the cosmetically acceptable salts or esters thereof.

It is nevertheless to be understood that in the composite material which can be used within the scope of the present invention, the metal derivative nanoparticles can be bound to at least one sun filter derivative, or only some of the nanoparticles of said metal derivative can be chemically bound to at least one sun filter derivative, the other metal atoms, which are not chemically bound to a sun filter derivative, then being present in the nanoparticle in oxidised form, preferably in the form of a metal oxide.

Schematically, and theoretically, the composite material is in the form of nanoparticles of metal derivative onto the surface of which at least one sun filter derivative is grafted; in particular, the nanoparticles have a surface which is substantially covered with sun filters, which are chemically bound to said nanoparticles of metal derivative.

It should also be understood that said sun filter derivatives are grafted onto the surface of the nanoparticles, and can nevertheless also be included in the nanoparticle itself.

The nanoparticles of the material which can be used within the scope of the present invention may in particular be obtained by means of the method of preparation described below in the present description, and for example by a similar process to that disclosed by S. Daniele et al. (J. Mater. Chem., 13, (2003), 342-346).

Therefore, the composite material of metal nanoparticles and organic sun filters can advantageously be obtained by inorganic polymerisation (sol-gel method), in a single step, from at least one hydrolysable precursor of a metal which has been modified with at least one organic sun filter.

By hydrolysable precursor of a metal is meant, for example, the alkoxides, amides or halides of one or more of the metals disclosed above, more specifically of the metals of which the oxidised forms have semiconductor properties. Preferably, the precursors are selected from metal alkoxides, more preferably from the alkoxides of the following formula (I):

[MOₓR′_{z}(AR″)_{w}(OR)_{(v-2x-z-w)}]ₘ (I)

in which:

M represents an atom of a metal advantageously selected from titanium, zinc, cerium, zirconium, and copper;

O represents an oxygen atom

v represents the valency of the metal M;

x is a number greater than or equal to zero and less than v/2; (0≦x≦v/2);

z is a number greater than or equal to zero and less than v; (0≦z≦v);

w is a number greater than zero and less than or equal to v; (0≦w≦v);

m is the rate of oligomerisation of the precursor of formula (I) and is an integer greater than or equal to 1, preferably from 1 to 100 inclusive;

2x+z+w≦v;

A represents a CO₂ or SO₃ group;

R is selected from a linear or branched alkyl radical containing 1 to 30 carbon atoms, a substituted or unsubstituted cycloalkyl radical containing 3 to 9 endocyclic carbon atoms, and a substituted or unsubstituted aryl radical containing 6 to 10 atoms;

R′ represents a halogen atom selected from fluorine, chlorine, bromine, iodine and astatine, or represents a hydroxyl group; and

R″ represents a sun-filter residue, comprising a group which absorbs ultraviolet rays in the wavelength range from 250 nm to 400 nm.

According to a particularly preferred embodiment, the metal is titanium. The compound of formula (I) becomes the compound of the following formula (I_{Ti})

[TiOₓR′_{z}(AR″)_{w}(OR)(4−2x−z−w)]ₘ (I_{Ti})

in which:

x is a number greater than or equal to zero and less than 2; (0≦x≦2);

z is a number greater than or equal to zero and less than 4; (0≦z≦4);

w is a number greater than zero and less than or equal to 4; (0≦w≦4);

2x+z+w≦4;

O, m, R′, R′ and R″ are as defined above;

According to an advantageous variant, the inorganic polymerisation of at least one hydrolysable precursor of formula (I) and/or (I_{Ti}) can be carried out in the presence of another hydrolysable compound, for example an alkoxide, amide or halide, as defined above, and in particular, of another hydrolysable metal compound which does not comprise any sun filter residue.

The simultaneous inorganic polymerisation (or hydrolysis) of precursors modified by at least one organic filter and of “pure” (i.e. unmodified) precursors, according to the method disclosed above, allows an alteration of the density of the organic sun filters which are grafted onto the surface of the metal derivative nanoparticle.

By way of non-limiting example, the inorganic polymerisation of at least one hydrolysable precursor of formula (I) can be carried out in the presence of another hydrolysable compound of the following formula (II):

[M′(OR)_{v′}]_{m′} (II)

in which:

M′ is an atom of a metal which is the same as or different from M, selected from titanium, zinc, cerium, zirconium, and copper;

m′ is the rate of oligomerisation of the compound (II) and is an integer greater than or equal to 1, preferably from 1 to 100 inclusive;

v′ is the valency of the metal M′; and

R is selected from a linear or branched alkyl radical containing 1 to 30 carbon atoms, a substituted or unsubstituted cycloalkyl radical containing 3 to 9 endocyclic carbon atoms, and a substituted or unsubstituted aryl radical containing 6 to 10 atoms;

When the metal is titanium, the compound of formula (II) becomes the compound of the following formula (II_{Ti}):

[Ti(OR)₄]_{m′} (II_{Ti})

in which R and m′ are as defined above.

The inorganic polymerisation referred to in the above may be carried out in a hydro-organic medium which is either mostly or purely water. The benefit of working in a purely aqueous medium is solely due to the concern of respecting the current rules and guidelines on the protection of the environment and on toxicity, particularly when the composite material may come into contact with living organisms, animals or humans, and in particular into contact with the skin.

When an organic solvent is present (inorganic polymerisation in a hydro-organic medium), said solvent is advantageously selected from the alcohols, preferably the mono-alcohols, and for example from methanol, ethanol, n-propanol, iso-propanol, n-butanol, iso-butanol and tert-butanol, and mixtures thereof in any proportions.

This list is not in any way limiting and other solvents may be used, such as unsaturated hydrocarbons, for example toluene, saturated hydrocarbons, for example hexane, aliphatic ethers or cyclic ethers, for example diethyl ether or tetrahydrofuran, as well as solvents of the dimethylformamide or dimethylsulphoxide type. Mixtures of two or more of the solvents mentioned here can also be used.

According to a preferred embodiment of the method, the inorganic polymerisation takes place in the presence of at least one ionic salt. The appropriate ionic salts can be selected from nitrates, halides, sulphates, or alkali, alkaline-earth or ammonium phosphates, and mixtures of two or more of these in any proportions. By way of non-limiting example, the ionic salt can be selected from potassium nitrate, lithium bromide, magnesium sulphate, and tetra-n-butylammonium bromide. A preferred ionic salt is tetra-n-butylammonium bromide.

The quantity of ionic salt which is used can vary greatly, and is generally between 0.5% and 20% by weight, preferably between 1% and 10% by weight, based on the original metal precursor.

The inorganic polymerisation reaction takes place when at least one hydrolysable metal precursor modified by at least one sun filter derivative as defined above, optionally in the presence of another unmodified hydrolysable precursor, comes into contact with water or a water/solvent mixture.

The amount of water which is necessary for the inorganic polymerisation reaction can also vary greatly, and in general 100 ml of water is used for a quantity of metal precursor(s) between 0.1 g and 10 g. The amount of organic solvent or of organic solvent mixture is generally between 0% and 20% by weight based on the amount of water used.

This inorganic polymerisation can be carried out at ambient temperature or at a temperature between the ambient temperature and the reflux temperature of the reaction medium, for example at a temperature between 15° C. and 150° C., advantageously between 25° C. and 130° C., preferably between 80° C. and 120° C., preferably at approximately 100° C.

However, the heating of the reaction medium is not necessary, but does promote the formation of nanometric particles in crystalline form, rather than amorphous form, i.e. the composite material obtained displays diffraction bands under X-ray radiation.

The inorganic polymerisation reaction is generally carried out at normal atmospheric pressure, for a duration varying from a few tens of minutes to a few hours, generally between one and three hours, for example two hours.

The solid is extracted from the reaction medium, by the conventional methods known to the person skilled in the art, for example by centrifuging, washed, rinsed, then dried.

This polymerisation reaction allows a solid to be obtained in a single step and in very moderate conditions, which solid is ready for use and is generally crystalline, in the form of nanometric particles, and is functionalised, i.e. has at least one sun filter derivative chemically bound thereto by covalent bonding.

This composite material is thus obtained in a very economical manner in a single step and in an aqueous or hydro-organic medium, starting from modified metal precursors which can easily be obtained, and in particular starting from alkoxides which have been modified with one or more organic sun filter derivatives of formula R″-A-Y, as defined above. It should be understood that when the organic sun filter is an ester (Y being a saturated or unsaturated, linear, branched or cyclic hydrocarbon group, containing 1 to 30 carbon atoms), said ester must be hydrolysed beforehand in acid or in salt, by conventional hydrolysis methods known to the person skilled in the art.

The aforementioned metal precursors are known, commercially available, or easily synthesised using known procedures which emerge from standard chemistry works, from publications, from patents, from “Chemical Abstracts”, or from the Internet.

Furthermore, the composite material which can be used within the scope of the present invention has a high functionalisation level and efficient adhesion, via covalent bonding, of the sun filter derivative, and therefore has a higher stability in any medium of which the pH is between 2 and 10.

By “functionalisation level” is meant the weight ratio of sun filter derivative to mineral component in the composite material. This functionalisation level is generally between 0.1% and 30%, preferably between 0.5% and 20%, advantageously between 1% and 15%, for example between 1.5% and 10%. Highly satisfactory results for the applications relevant to the present invention have been obtained with functionalisation levels between 2% and 5% by weight.

The composite material which can be used within the scope of the present invention takes the form of nanometric particles such as have just been defined, which have the advantage of being dispersible in a manner compatible with the application according to the present invention. In fact, good dispersion of the nanoparticles demonstrates low aggregation between the nanoparticles, as well as a relatively low agglomerate particle size.

Furthermore, good dispersion is necessary for the formulation of the composite material in the cosmetic preparations relevant to the present invention, in particular sun products, care products and make-up products.

The inventors have demonstrated that the dispersion varies depending on the functionalisation level defined above, and on the nature of the sun filter grafted onto the nanoparticles.

In general, the composite materials of the present invention have an agglomerate particle size between 100 nm and 3000 nm, most often between 150 nm and 2500 nm, in particular between 160 nm and 2200 nm, for functionalisation levels of about 1.5% to 8%.

The inventors have surprisingly found that the composite material defined above has highly beneficial and unexpected properties when used as a sun filter incorporated into compositions for topical application to the dermis, for example in the form of a cream, oil, gel, spray, lotion, powder and so on.

In fact, the material comprises organic sun filters bound to metal derivative particles, for example semiconductor titanium oxide particles, by a strong chemical bond, thus avoiding the freeing of said sun filters into the surrounding medium.

The size of the nanoparticles in the composite material also means that said mixture is kept on the surface of the skin without being able to pass through.

Therefore, after application of the composite material to the skin, the organic sun filter is retained on the skin surface with the metal derivative particles. While it remains bound to said nanoparticles, there is no risk of it penetrating through the skin and subsequently diffusing into the blood stream.

Another surprising feature is that the inherent toxicity of the nanoparticles of metal derivatives is considerably reduced because the protection of the organic sun filters makes said nanoparticles much less open to UV radiation.

Finally, it has surprisingly been found that the composite material as defined above has the feature of increasing the range of wavelengths absorbed to include longer wavelengths (UV-A) as far as 450 nm, thus giving a significant and unexpected width to the spectrum of protection against ultraviolet radiation.

In particular, the use of nanoparticles of nanometric size which are based on titanium allows the phenomenon of light diffusion, and thus of whitening of the skin upon application of sun cream, to be avoided. These nanoparticles also have the advantage that they can be dispersed homogeneously and thus diminish the quantity by weight of active ingredient, whilst improving the texture of the cosmetic formulations obtained.

Therefore, the composite materials of the present invention have an application which is highly suitable in cosmetics, as UV filters forming part of the composition of protective products, in particular for the skin, against UV rays.

In accordance with the intended applications, the composite materials of the invention can be used alone or in combination with the photostabilised metal oxide particles which are commonly used, in particular titanium dioxide, and even with conventional organic filters.

The present invention also relates to cosmetic compositions which comprise an effective amount of at least one composite material as defined above, alone or in combination with one or more other organic or inorganic sun filters which are known from the prior art, in a cosmetically acceptable medium, such as for example a medium comprising one or more vehicles, fillers, textural agents, colours, pigments, emollients and so on, commonly used in the field of cosmetology.

The composite material as defined above is generally incorporated into cosmetic compositions in sufficient concentrations to absorb ultraviolet rays and in cosmetically acceptable concentrations. These concentrations are generally between 0.01% and 50% by weight based on the total weight of the composition, more preferably between 0.05% and 30%, and even more preferably between 1% and 20%.

The formulations comprising at least one composite material according to the present invention are thus an integral part of the present invention. The formulations may be in various forms which are known in the field and for example in the form of a cream, oil, gel, spray, lotion, powder, and so on.

The cosmetic compositions defined above are useful in particular for protecting the skin against ultraviolet (UV) radiation, both UV-A and UV-B, and offer a spectrum of protection of which the width covers the whole range of ultraviolet radiation wavelengths, that is from 250 nm to 400 nm, and surprisingly provides a significant, non-negligible absorption at around 400 nm.

The present invention therefore provides a very effective means for protecting the skin against ultraviolet radiation, whilst minimising the known undesirable effects connected with the metal particles on the one hand and the organic sun filters on the other.

The following examples are given purely by way of illustration and are not of a limiting character.

### EXAMPLE 1

### Preparation of a Composite TiO₂/Para-Aminobenzoic Acid Material

Starting from titanium para-aminobenzoate tri-isopropoxide in an aqueous medium

| | |
|---|---|
| [Ti(OC₃H₇)₃(O₂CC₆H₄NH₂)]ₘ | 0.90 g |
| Tetra-n-butylammonium bromide (NⁿBu₄Br) | 0.08 g |
| Water (H₂O) | 50.00 g |

The [Ti(OC₃H₇)₃(O₂CC₆H₄NH₂)]ₘ is obtained by an equimolar reaction between the titanium tetra-isopropoxide [Ti(OC₃H₇)₄] and the para-aminobenzoic acid (HO₂CC₆H₄NH₂, PABA). The index m represents the number of precursors involved in a crystal lattice.

The [Ti(OC₃H₇)₃(O₂CC₆H₄NH₂)]ₘ is added into the aqueous solution of NⁿBu₄Br which has been brought to reflux, and is stirred for 2 hours while maintaining the reflux. The solid is recovered by centrifuging then washed in water and in ethanol. After drying at 70° C. for 20 hours, the material is ready for use. The material has a functionalisation level of 7.6% by weight.

### EXAMPLE 2

### Preparation of a Composite TiO₂/PABA Material

Starting from Titanium Para-Aminobenzoate Tri-Isopropoxide, in a Hydro-Organic Medium which is Mostly Water, in the Presence of Titanium Alkoxide

| | |
|---|---|
| [Ti(OC₃H₇)₃(O₂CC₆H₄NH₂)]ₘ | 0.13 g |
| Titanium tetra-isopropoxide [Ti(OC₃H₇)₄]ₘ | 6.75 g |
| Tetra-n-butylammonium bromide (NⁿBu₄Br) | 0.80 g |
| Isopropanol (HOC₃H₇) | 6.00 g |
| Water (H₂O) | 75.00 g |

The isopropanol solution comprising [Ti(OC₃H₇)₃(O₂CC₆H₄NH₂)]ₘ+[Ti(OC₃H₇)₄]ₘ is added into the aqueous solution of NⁿBu₄Br which has been brought to reflux, and is stirred for 3 hours while maintaining the reflux. The solid is recovered by centrifuging then washed in water and in ethanol. After drying at 70° C. for 20 hours, the material is ready for use. The material has a functionalisation level of 1.5% by weight.

### EXAMPLE 3

### Preparation of a Composite TiO₂/Uvinul MS 40® Material

Starting from Titanium 5-Benzoyl-4-Hydroxy-2-Methoxybenzene Sulphonate-Tri-Isopropoxide in an Aqueous Medium

| | |
|---|---|
| [Ti(OC₃H₇)₃(C₁₄H₁₂O₆S)]ₘ | 0.18 g |
| Tetra-n-butylammonium bromide (NⁿBu₄Br) | 0.01 g |
| Water (H₂O) | 50.00 g |

The [Ti(OC₃H₇)₃(C₁₄H₁₂O₆S)]ₘ is obtained by an equimolar reaction between the titanium tetra-isopropoxide [Ti(OC₃H₇)₄] and the 5-benzoyl-4-hydroxy-2-methoxybenzene sulphonic acid (Uvinul MS 40®).

The [Ti(OC₃H₇)₃(C₁₄H₁₂O₆S)]ₘ is added into the aqueous solution of NⁿBu₄Br which has been brought to reflux, and is stirred for 2 hours while maintaining the reflux. The solid is recovered by centrifuging then washed in water and in ethanol. After drying at 70° C. for 20 hours, the material is ready for use.

### EXAMPLE 4

### Preparation of a Composite TiO₂/Uvinul MS 400 Material

Starting from Titanium 5-Benzoyl-4-Hydroxy-2-Methoxybenzene Sulphonate-Tri-Isopropoxide in an Aqueous Medium which is Mostly Water, in the Presence of Titanium Alkoxide

| | |
|---|---|
| [Ti(OC₃H₇)₃(C₁₄H₁₂O₆S)]ₘ | 0.1 g |
| Titanium tetra-isopropoxide [Ti(OC₃H₇)₄]ₘ | 0.8 g |
| Tetra-n-butylammonium bromide (NⁿBu₄Br) | 0.1 g |
| Isopropanol (HOC₃H₇) | 6.0 g |
| Water (H₂O) | 75.0 g |

The isopropanol solution comprising [Ti(OC₃H₇)₃(C₁₄H₁₂O₆S)]ₘ+[Ti(OC₃H₇)₄]ₘ is added into the aqueous solution of NⁿBu₄Br which has been brought to reflux, and is stirred for 3 hours while maintaining the reflux. The solid is recovered by centrifuging then washed in water and in ethanol. After drying at 70° C. for 20 hours, the material is ready for use.

All these materials have been unequivocally characterised by elemental analyses, infrared spectroscopies with Fourier transformations and visible UV, diffraction of X-rays on powder, transmission electron microscopy and photoelectronic spectroscopy.

### EXAMPLE 5

### Example Formulation: Foundation

The quantities are given as percentages by weight

| | |
|---|---|
| Modified polysiloxane polyether | 3.00 |
| Sorbitan-9-octadecanoate-2,3 | 1.00 |
| Isododecane | 8.00 |
| Pentacyclomethicone | 18.51 |
| Mineral pigments | 10.00 |
| Phenoxyethanol | 0.30 |
| Bentone gel VS 5PC V | 3.00 |
| Water | 44.62 |
| Tetrasodium EDTA | 0.10 |
| Sodium chloride | 2.00 |
| Butylene glycol-1,3 | 5.00 |
| Methylparahydroxybenzoate | 0.20 |
| Chlorophenesin | 0.27 |
| Composite TiO₂/Uvinul MS 40 ® material, | 4.00 |
| according to example 3 | |

### EXAMPLE 6

### Example Formulation: Sun Cream

The quantities are given as percentages by weight

| | |
|---|---|
| Pentacyclomethicone | 40.65 |
| Laurylmethicone copolyol | 3.00 |
| Phenyltrimethylpolysiloxane | 7.50 |
| Octyl methoxycinnamate | 10.00 |
| Mixed phenoxyethanol paraben | 0.60 |
| DL-α-tocopherol acetate | 0.50 |
| Water | 18.00 |
| Butylene glycol-1,3 | 5.00 |
| Composite TiO₂/Uvinul MS 40 ® material, | 14.75 |
| according to example 4 | |

### EXAMPLE 7

### Example Formulation: Anti-Wrinkle, Moisturising Day Cream, in the Form of an Oil-in-Water Emulsion

The quantities are given as percentages by weight.

### Phase A:

| | |
|---|---|
| Water | 57.5 |
| Methylparaben | 0.1 |
| Chlorphenesin | 0.3 |
| Phenoxyethanol | 0.4 |
| Acrylates/C10-30 Alkyl acrylate crosspolymers | 0.5 |
| Glycerol | 3.0 |
| Composite TiO₂/PABA material, | 10.0 |
| according to example 2 | |
| 1,3-Butylene glycol | 3.0 |

### Phase B:

| | |
|---|---|
| Steareth-2 | 1.3 |
| Steareth-21 | 2.2 |
| Glyceryl stearate | 1.0 |
| Cetylic alcohol | 2.2 |
| Stearic alcohol | 2.2 |
| Stearin 50/50 | 1.8 |
| Cetyl palmitate | 1.3 |
| Hydrogen polyisobutene | 12.3 |
| Preservatives | 0.7 |

### Phase C:

| | |
|---|---|
| DL-α-tocopherol acetate | 0.2 |

Notes:
“Acrylates/C10-30 Alkyl Acrylate Crosspolymers” are copolymers formed from a C₁₀-C₃₀ alkyl acrylate, with one or more monomers of acrylic acid, of methacrylic acid or of one of the esters thereof, cross-linked with an allylic ether of sucrose or an allylic ether of pentaerythritol. Copolymers of this type are commercially available, in particular, from Noveon, Inc., USA.
Stearin 50/50 is a 50:50 mixture by weight of hexadecanoic acid and octadecanoic acid.
The “preservatives” which appear in Phase B above consist of a mixture of methylic, ethylic, propylic, butylic and isobutylic esters, commercially available from Clariant Corp., USA.

The following description by way of reference to the attached figures will allow the subject-matter of the invention to be better understood. Where they are not mutually exclusive, the various embodiments described in the following may be combined.

FIG. 1 is an infrared spectrum with Fourier transformation of a composite material of titanium nanoparticles grafted by Uvinul MS-40® (5-benzoyl-4-hydroxy-2-methoxybenzenesulphonic acid).

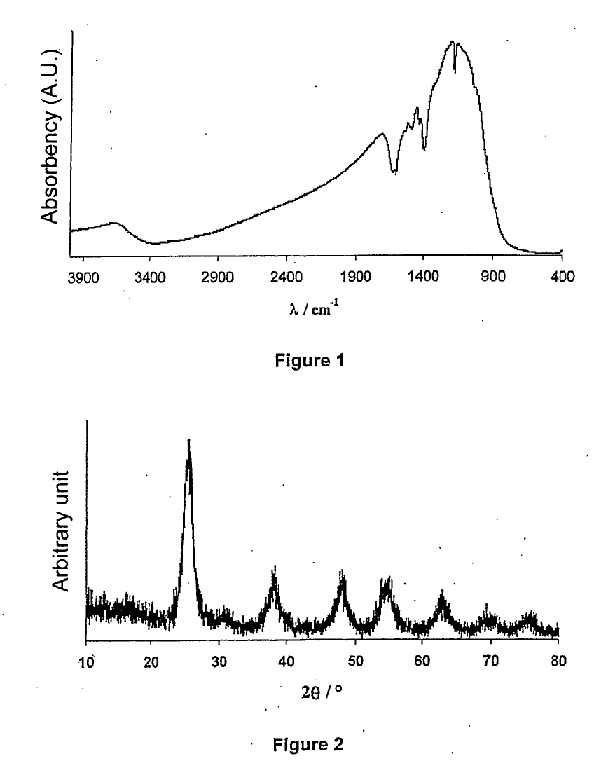

FIG. 2 is a diffraction spectrum of X rays on a composite material of titanium nanoparticles grafted by para-aminobenzoic acid (PABA).

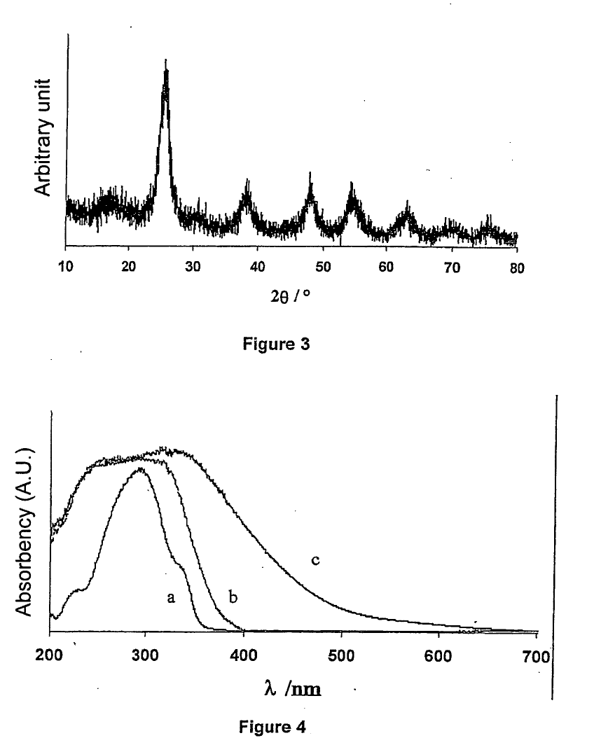

FIG. 3 is a diffraction spectrum of X rays on a composite material of titanium nanoparticles grafted by Uvinul MS-40® (5-benzoyl-4-hydroxy-2-methoxybenzenesulphonic acid).

FIG. 4 shows a comparison of the UV absorption spectra in the solid state of a) para-aminobenzoic acid alone, b) titanium dioxide particles (Degussa P25) and c) the composite material consisting of titanium nanoparticles grafted by para-aminobenzoic acid (PABA).

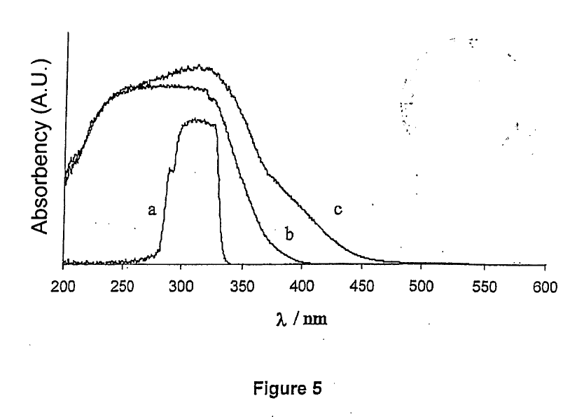

FIG. 5 shows a comparison of the UV absorption spectra in the solid state of a) Uvinul MS-40® (5-benzoyl-4-hydroxy-2-methoxybenzenesulphonic acid), b) titanium dioxide particles (Degussa P25) and c) the composite material consisting of titanium nanoparticles grafted by Uvinul MS-40® (5-benzoyl-4-hydroxy-2-methoxybenzenesulphonic acid).

From line c of FIGS. 4 and 5, a displacement of the absorbed wavelengths by comparison with the organic sun filters alone (line a) and with the inorganic sun filters alone (line b) can be seen clearly.

1. A method of providing protection from ultraviolet rays, comprising applying to a surface to be protected a composite material comprising:
nanoparticles of at least one metal derivative and
at least one organic sun-filter derivative, covalently chemically bound to said nanoparticles,
in the form of an ultraviolet ray protective agent.

2. Method according to claim 1, wherein said sun-filter derivative is covalently chemically bound to said metal derivative.

3. Method according to claim 1, wherein said metal derivative has semiconductor properties.

4. Method according to claim 1, wherein said metal derivative is capable of absorbing ultraviolet rays.

5. Method according to claim 1, wherein the metal of said metal derivative is selected from the group consisting of titanium, zinc, cerium, zirconium, copper, and mixtures thereof.

6. Method according to claim 1, wherein the metal of said metal derivative is selected from the group consisting of titanium, zinc, cerium, and mixtures thereof.

7. Method according to claim 1, wherein said metal derivative is a metal oxide or a mixture of metal oxides.

8. Method according to claim 7, wherein said metal oxide and the metal oxides of said mixture are selected from the group consisting of titanium dioxide (TiO2), zinc monoxide (ZnO), cerium oxides (Ce2O3 and CeO2), zirconium dioxide (ZrO2) and copper oxides (CuO and Cu2O).

9. Method according to claim 7, wherein said metal oxide and the metal oxides of said mixture are selected from the group consisting of titanium dioxide (TiO2), zinc monoxide (ZnO), and cerium oxides (Ce2O3 and CeO2).

10. Method according to claim 1, wherein said nanoparticles have a mean diameter of between 2 nm and 50 nm.

11. Method according to claim 1, wherein the sun filter is selected from those sun filters which absorb UV rays in the wavelength range from 250 nm to 400 nm.

12. Method according to claim 1, wherein the sun filter contains at least one carboxylic acid functional group and/or sulphonic acid functional group, said functional groups being either converted to the salt thereof or not, or being either converted to the ester thereof or not.

13. Method according to claim 1, wherein the sun filter has the formula:
R″-A-Y
in which R″ represents a sun-filter residue, comprising a group which absorbs ultraviolet rays in the wavelength range from 250 nm to 400 nm,
A-Y represents a carboxylic group COOY or sulphonic group SO3Y, where Y is selected from:
a hydrogen atom;
a cation of an alkali metal or of an alkaline-earth metal from the periodic table;
a cation derived from a primary, secondary, tertiary or quaternary amine, or an ammonium cation; and
a saturated or unsaturated, linear, branched or cyclic hydrocarbon group, containing 1 to 30 carbon atoms.

14. Method according to claim 1, wherein the sun filter is selected from:
4-aminobenzoic acid;
4-dialkylaminobenzoic acids of which the alkyl groups, optionally carrying a hydroxyl group, are C1-C8 alkyl groups;
2-phenylbenzimidazole-5-sulfonic acid;
3,3′-(1,4-phenylidenedimethylidene-bis-(7,7-dimethyl-2-oxobicyclo[2,2,1]-hept-1-ylmethanesulphonic) acid or benzylidene-camphor sulphonic acid;
α-(oxo-2-bornylidene-3)-toluene-4-sulphonic acid or terephthalylidene-camphor sulphonic acid;
2-hydroxy-4-methoxybenzophenone-5-sulphonic acid;
2,2′-(1,4-phenylene)-bis-1H-benzimidazole-4,6-disulphonic acid;
salicylic acid;
4-alkoxycinnamic acids of which the alkoxy group is a C1-C10 alkoxy group;
3,3′-carbonyl-bis-(4-hydroxy-6-methoxy)benzene sulphonic acid;
2-cyano-3,3-diphenylacrylic acid; and
anthranylic acid
as well as cosmetically acceptable salts or esters thereof.

15. Method according to claim 13, wherein the residue R″ comprises at least one benzene ring and/or at least one benzimidazole ring and/or at least one bornyl ring.

16. Method according to claim 15, wherein said acid functional groups are carried on an aromatic ring.

17. Method according to claim 15, wherein the sun filter is selected from the group consisting of para-aminobenzoic acid, 5-benzoyl-4-hydroxy-2-methoxybenzenesulphonic acid, 2-phenylbenzimadole-5-sulphonic acid, 3,3′-(1,4-phenylidenedimethylidene)bis-(7,7-dimethyl-2-oxobicyclo[2,2,1]hept-1-yl-methane-sulphonic) acid and salts thereof, α-(oxo-2-bornylidene-3)-toluene-4-sulphonic acid and salts thereof, 2-ethylhexyl 2-cyano-3,3-diphénylacrylate, octyl 4-methoxycinnamate, and ethoxylated ethyl 4-aminobenzoate, and cosmetically acceptable salts or esters thereof.

18. Method according claim 1, wherein the composite material comprises titanium dioxide nanoparticles which are chemically grafted, by covalent bonding, to at least one sun-filter derivative, by at least one carboxylic and/or sulphonic functional group.

19. Method according to claim 1, wherein the composite material comprises titanium dioxide nanoparticles which are chemically grafted, by covalent bonding, to at least one sun-filter derivative, by at least one carboxylic and/or sulphonic functional group, said sun filter being selected from the group consisting of para-aminobenzoic acid, 5-benzoyl-4-hydroxy-2-methoxybenzenesulphonic acid, 2-phenylbenzimidazole-5-sulphonic acid, and the cosmetically acceptable salts or esters thereof.

20. Cosmetic composition comprising an effective amount of at least one composite material comprising nanoparticles of at least one metal derivative and at least one organic sun-filter derivative, covalently chemically bound to said nanoparticles, alone or in combination with one or more other organic and/or inorganic filters, in a cosmetically acceptable medium.

21. Cosmetic composition according to claim 20, in the form of a cream, oil, gel, spray, lotion, or powder.
